(19) United States
(12) Patent Application Publication
(10) Pub. No.: US 2019/0030315 A1
(43) Pub. Date: Jan. 31, 2019

(54) Hose Clamp for Clamping Shut a Medical Hose

(71) Applicant: B. BRAUN MELSUNGEN AG, MELSUNGEN (DE)

(72) Inventors: Michael Fiege, Kassel (DE); Gerrit Seidel, Kassel (DE); Christine Ton-That, Melsungen (DE)

(21) Appl. No.: 16/033,908

(22) Filed: Jul. 12, 2018

(30) Foreign Application Priority Data
Jul. 26, 2017 (DE) 102017212883.6

(51) Int. Cl. A61M 39/28; A61M 5/168

(52) U.S. Cl. CPC A61M 39/287; A61M 2205/3331; A61M 39/284; A61M 5/16813

(57) ABSTRACT

A hose clamp for clamping shut a medical hose includes two clamping legs. In relation to a duct transverse direction in each case, one delimiting element is disposed on opposite transverse sides of a passage duct. The delimiting elements are designed in such a manner that the passage duct in the region of the clamping faces is closed in the duct transverse direction. The passage duct in the duct transverse direction is at least in portions released both ahead of and behind the clamping faces in relation to the duct longitudinal direction, such that the hose to be clamped shut, when viewed in a lateral direction, remains uncovered by the delimiting elements across as wide a region of the passage duct as possible. The hose clamp can be used in infusion therapy.

### RELATED APPLICATION(S)

This application is related to and claims the benefit of priority of German Patent Application DE 10 2017 212 883.6, the content of which is incorporated by reference in its entirety into this application.

### FIELD

The disclosure relates to a hose clamp for clamping shut a medical hose, having two clamping legs which are mutually movable about a pivot axis between an opening position and a clamped-shut position and in the clamped-shut position are mutually latched by a, a passage duct for receiving the hose to be clamped shut, wherein the passage duct along a duct longitudinal direction extends between two passage openings and in relation to a duct vertical direction on an upper side and a lower side is in each case at least in portions covered by the clamping legs, and two clamping elements which are disposed on the upper side and the lower side of the passage duct and which have in each case one clamping face, the latter by way of the movement of the clamping legs being mutually actuatable to the clamped-shut position in such a manner that the passage duct in the duct vertical direction is capable of being constricted and the hose that is receivable in the passage duct is capable of being clamped shut.

### BACKGROUND

A hose clamp is known from EP 0 517 000 A2. The known hose clamp has a bracket-shaped main body having an upper clamping leg and a lower clamping leg, each in the form of a bracket leg, and two end-side passage openings. A passage duct which is provided for receiving a medical hose that is to be clamped shut extends in the vertical direction between the bracket legs. A clamping element in the form of a wedge-shaped clamping jaw is molded in each case to the inside of the two bracket legs, said clamping element having in each case one substantially bead-type clamping face. In order for the hose to be clamped shut, the bracket legs are mutually moved in a known manner by means of finger pressure and are mutually latched by means of a latching means having a latching tongue and a latching step. That portion of the passage duct that lies between the clamping faces is constricted in the duct vertical direction in such a manner that a hose that is disposed in the passage duct is capable of being clamped shut. In addition to the main body, a separate insert body which can be clipped between the two bracket legs is provided. This insert body is intended to prevent the hose to be clamped shut being bulged in the transverse direction beyond the clamping faces and, as a result thereof, not being clamped or only being inadequately clamped.

It is an object of this disclosure to provide a hose clamp of the type mentioned at the outset which has improved properties.

### SUMMARY

This object is achieved in that in relation to a duct transverse direction in each case one delimiting element is disposed on opposite transverse sides of the passage duct, wherein the delimiting elements are designed in such a manner that the passage duct in the region of the clamping faces is closed in the duct transverse direction and said passage duct in the duct transverse direction is at least in portions released both ahead of and behind the clamping faces in relation to the duct longitudinal direction, such that the hose to be clamped shut—when viewed in a lateral direction—remains uncovered by the delimiting elements across as wide a region of the passage duct as possible. On account of the solution according to the disclosure, the passage duct in the region of the clamping faces is closed in the duct transverse direction, on the one hand. This prevents the hose to be clamped shut being able to bulge outwards laterally between the clamping faces and as a result thereof not being clamped or being only partially clamped. A functionally reliable clamping is guaranteed in this way. On the other hand, it is achieved on account of the solution according to the disclosure that the passage duct in the duct transverse direction is released ahead of and behind the clamping faces in relation to the duct longitudinal direction. In other words, the hose to be clamped shut when viewed in a lateral direction remains uncovered by the delimiting elements across as wide a region of the passage duct as possible. In this way, the hose to be clamped shut, despite the closure according to the disclosure in the transverse direction by means of the delimiting elements, remains readily viewable from the outside. Medical personnel can consequently check in a simple way whether or not an orderly clamping of the hose has been achieved. The clamping elements can in each case be designed in the form of wedge-shaped or rounded clamping jaws. The clamping faces can advantageously be designed so as to be convex, bead-shaped, or in the manner of a circular cylindrical face. The latching means can advantageously comprise a latching cam and a latching hook which in the clamped-shut position of the hose clamp are mutually latched. Both the latching cam as well as the latching hook can in each case be disposed on an end side of one of the clamping legs, particularly be integrally molded thereon. The delimiting elements can in each case be disposed on the upper one or the lower one of the clamping legs in relation to the duct vertical direction. Alternatively, in each case one of the delimiting elements can be disposed on both of the clamping legs.

In one design embodiment, the delimiting elements in the duct longitudinal direction extend in each case approximately across the length of the clamping elements, preferably across the length of the clamping faces. In the case of this design embodiment of the disclosure there is now as before sufficient support in the duct transverse direction of the hose to be clamped shut such that said hose is prevented from laterally bulging between the clamping faces. On the other hand, a particularly good visibility of the hose to be clamped shut when viewed in the lateral direction is achieved on account of this type of the dimensioning of the length of the delimiting elements.

In a further design embodiment, the delimiting elements in the duct longitudinal direction extend in each case across at most double the length of the clamping faces, preferably across at most 1.5 times the length of the latter. This design embodiment achieved an even further improved visibility of the hose to be clamped shut when viewed in the lateral direction. Moreover, material can be saved and thus a cost-effective production can be achieved by way of a design embodiment of the delimiting elements that is restricted in length in this manner.

In a further design embodiment, the delimiting elements are in each case configured in the form of a strip that is oriented substantially in the duct vertical direction. The delimiting elements that are configured so as to be strip-shaped in such a manner can in each case have a rounded feature on the lower end side regions in relation to the duct vertical direction. In as far as a hose to be clamped shut inadvertently makes its way between one of the delimiting elements and one of the clamping elements, damage to the hose can be avoided as a result of the rounded feature.

In a further design embodiment, the delimiting elements have in each case one front edge and one rear edge which run in a mutually parallel or wedge-shaped manner. Accordingly, a particularly slim design of the delimiting elements is achieved, on account of which the lateral visibility of the hose to be clamped shut is yet again improved.

In a further design embodiment, the delimiting elements in the duct transverse direction are in each case flush with the face, or recessed, in relation to in each case one outside wall portion of one of the clamping legs. On account of this design embodiment, a hose clamp having end faces that have smooth surfaces on both sides, or without lateral protrusions, respectively, is achieved. For applications in which the hose clamp is in contact with the skin of a patient, an improved comfort is consequently achieved and pressure points are avoided.

In a further design embodiment, the delimiting elements are disposed on the upper one of the clamping legs in relation to the duct vertical direction. In as far as the upper clamping leg on the end side has a latching cam of the latching means, an improved production capability of the hose clamp can be achieved in certain circumstances on account of an arrangement of this type of the delimiting elements.

In a further design embodiment, the delimiting elements in an alternating manner are disposed on the upper clamping leg or on the lower clamping leg. This design embodiment provides the one delimiting element on the upper clamping leg, and the other delimiting element in an offset manner, opposite to the former, on the lower clamping leg. This design embodiment enables a simplified production capability.

In a further design embodiment, the delimiting elements are configured so as to be integral to the clamping legs and to the clamping elements. To this extent it is particularly advantageous for the hose clamp to be configured as an integral component, in particular as a plastic injection-molded component. This design embodiment permits a particularly cost-effective production, on the one hand. On the other hand, a compact construction mode is achieved on account of the molding of the delimiting elements so as to be integral to the further construction elements and/or molded elements of the hose clamp. Moreover, any unintentional release of the delimiting elements from the hose clamp is precluded in this manner, this, for example, in the case of a design embodiment of the delimiting elements in the form of an insert body not being able to be excluded in all circumstances.

In a further design embodiment, the extent of the delimiting elements in the duct vertical direction corresponds to at least the height of the passage duct between the clamping faces when the clamping legs are in the opening position. In other words, the delimiting elements are designed in such a manner that the passage duct in the region of the clamping faces is also closed in the duct transverse direction when the clamping legs assume the opening position. This is a design embodiment which is particularly functionally reliable, since lateral bulging of the hose to be clamped shut is counteracted in this manner also in the opening position of the clamping legs.

In a further design embodiment, the delimiting elements have in each case one wall portion which is in each case supported in the duct transverse direction in relation to one sliding wall portion of one of the clamping legs and/or of the clamping elements and in a sliding manner is movable perpendicular in relation to the duct transverse direction. The wall portions of the delimiting elements can in each case advantageously be a wall portion that faces the passage duct. Accordingly, the sliding wall portion can advantageously be a wall portion of one of the clamping legs and/or one of the clamping elements that faces away from the passage duct. Improved guiding of the clamping legs in a movement between the opening position and the clamped-shut position is achieved on account of such support of the delimiting elements in the transverse direction. Unintentional canting of the hose clamp when activated can be counteracted in this way.

### BRIEF DESCRIPTION OF THE DRAWING FIGURES

Further advantages and features of the disclosure are derived from the description hereunder of exemplary embodiments which are illustrated by means of the drawing figures, of which:

FIGS. 1 to 3 show an embodiment of a hose clamp according to the disclosure in an opening position in a schematic perspective illustration (FIG. 1), a side view (FIG. 2), and in a sectional illustration (FIG. 3) in a section plane B-B according to FIG. 2;

FIGS. 4 to 6 show the hose clamp as per FIGS. 1 to 3 in a clamped-shut position in a schematic perspective illustration (FIG. 4), in a side view (FIG. 5), and in a sectional illustration (FIG. 6) along a sectional plane A-A according to FIG. 5; and

FIGS. 7 to 9 show a further embodiment of a hose clamp according to the disclosure, similar to that of FIGS. 4 to 6.

### DETAILED DESCRIPTION

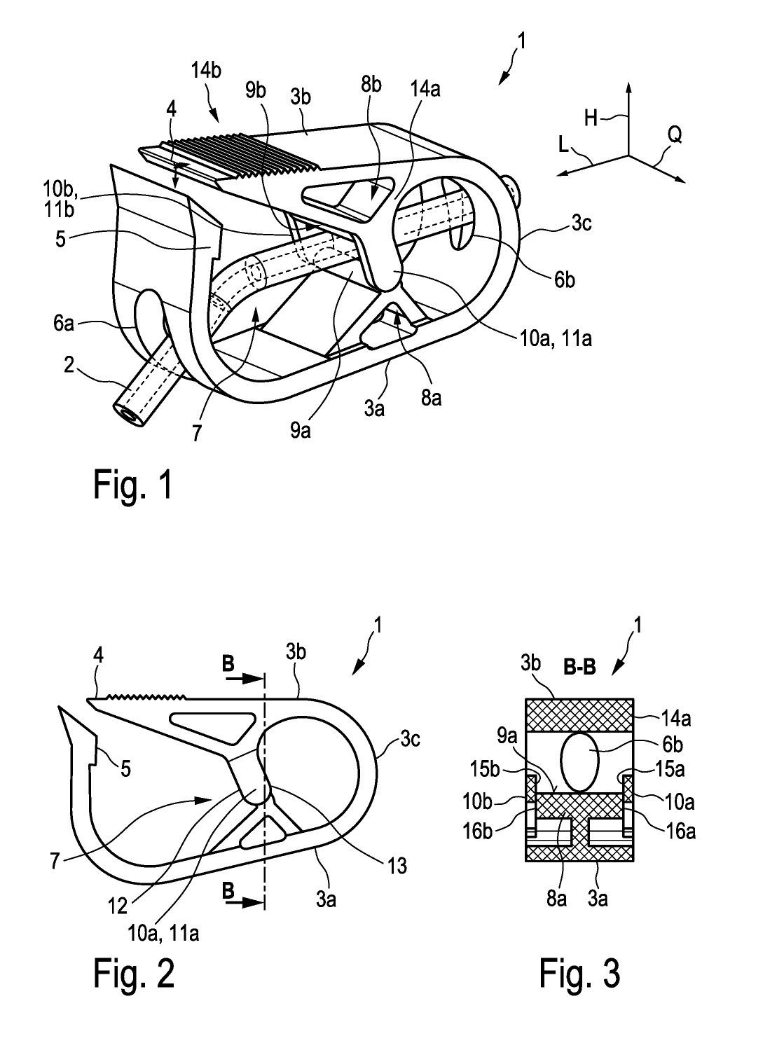

A hose clamp 1 as per FIGS. 1 to 6 is provided for clamping shut a medical hose 2 (FIG. 1) in particular in an infusion therapy.

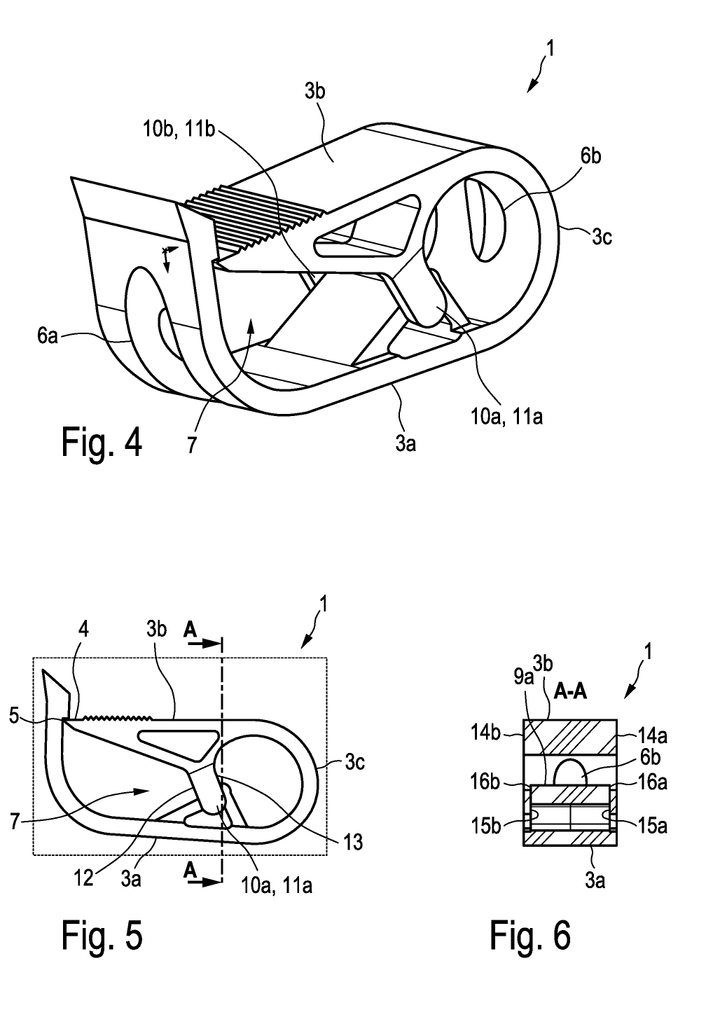

The hose clamp 1 has two clamping legs 3a, 3b. The two clamping legs 3a, 3b are interconnected by way of an arcuate flexible wall portion 3c and to this extent are mutually movable about a pivot axis (not indicated in more detail) between an opening position (FIGS. 1 to 3) and a clamped-shut position (FIGS. 4 to 6). The in relation to the image plane of FIGS. 1 to 6 upper clamping leg 3b on the end side has a latching cam 4. Accordingly, the lower clamping leg 3a on the end side has a latching hook 5. The latching cam 4 and the latching hook 5 form a latching means 4, 5 of the hose clamp 1, by way of which the clamping legs 3a, 3b in the clamped-shut position are mutually latched. The hose clamp 1 furthermore has a forward passage opening 6a and a rearward passage opening 6b. The passage openings 6a, 6b are in each case configured in the form of a substantially elliptic passage and are disposed in the wall of the lower clamping leg 3a, or the wall of the upper clamping leg 3b, respectively, and of the arcuate wall portion 3c. A passage duct 7 for receiving the hose 2 to be clamped shut extends between the passage openings 6a, 6b at least in portions along a duct longitudinal direction L. The passage duct 7 in relation to a duct vertical direction H is at least in portions covered by the clamping legs 3a, 3b on an upper side and a lower side. Moreover, the hose clamp 1 has clamping elements 8a, 8b that are disposed on the wall portions of the clamping legs 3a, 3b that face the passage duct 7. The clamping elements 8a, 8b in relation to the duct vertical direction H are disposed on opposite sides of the passage duct 7 and in each case have one clamping face 9a, 9b. The clamping elements 8a, 8b are in each case configured in the form of a clamping jaw that is designed so as to be wedge-shaped. The clamping faces 9a, 9b have in each case the shape of a cylindrical sleeve portion and to this extent are designed so as to be convex or bead-shaped. The clamping faces 9a, 9b are mutually actuatable by way of a movement of the clamping legs 3a, 3b to the clamped-shut position, said movement being activatable in a known manner by finger pressure onto the external faces of the clamping legs 3a, 3b. As a consequence of this actuation, the passage duct 7 in the region of the clamping faces 9a, 9b is capable of being constricted in the duct vertical direction H, the hose 2 to be clamped shut thus being capable of being clamped shut on the hose sleeve portion that is disposed between the clamping faces 9a, 9b. The hose clamp 1 in this way forms a type of blocking member having two functional positions (opened, closed) for a volumetric flow that flows through the hose 2.

The hose clamp 1 furthermore has two delimiting elements 10a, 10b. The delimiting elements 10a, 10b in relation to a duct transverse direction Q are disposed on opposite transverse sides of the hose clamp 1 and to this extent on opposite transverse sides of the passage duct 7. The delimiting elements 10a, 10b are designed in such a manner that the passage duct 7 in the region of the clamping faces 9a, 9b is closed in the duct transverse direction Q. Any unintentional lateral bulging of the hose 2 to be clamped shut and thus bypassing or an only partial clamping shut of the hose 2 is counteracted in this way. Moreover, the delimiting elements 10a, 10b are designed in such a manner that the passage duct 7 is at least in portions released in the duct transverse direction Q ahead of as well as behind the clamping faces 9a, 9b in relation to the duct longitudinal direction L. This guarantees a good visibility of the clamping region between the clamping faces 9a, 9b such that medical personnel can readily check a functionally correct clamping of the hose 2 at any time.

The delimiting elements 10a, 10b are configured so as to be integral to the clamping legs 3a and 3b, and to the clamping elements 8a and 8b, such that an integral embodiment of the hose clamp 1 is achieved. The hose clamp 1 can preferably be produced as a plastic injection-molded component.

The delimiting elements 10a, 10b are furthermore in each case configured in the form of a strip 11a, 11b. Said strips 11a, 11b are in each case disposed as a type of appendage on the opposite sides of the clamping element 8b and thus on the upper clamping leg 3b.

In relation to the duct longitudinal direction L the delimiting elements 10a, 10b extend in each case approximately across the length of the clamping faces 9a, 9b. In as far as the clamping elements 8a, 8b instead of the wedge-shaped design embodiment are configured as vertical wall portions, it can be sufficient for the delimiting elements 10a, 10b in the duct longitudinal direction L to in each case extend approximately across the length of the clamping elements 8a, 8b. The respective length of the delimiting elements 10a, 10b presently is in any case not more than 1.5 times the length of the clamping faces 9a, 9b. A particularly good visibility of the hose to be clamped shut in the region of the clamping faces 9a, 9b is achieved in this manner.

The delimiting elements 10a, 10b have in each case one front edge 12 and one rear edge 13. The front edge 12 and the rear edge 13 run so as to be substantially mutually parallel, wherein edge profiles that deviate therefrom are of course also possible. The front edge 12 and the rear edge 13 can particularly preferably run in the mutually wedge-shaped manner, wherein the spacing between the front edge 12 and the rear edge 13 tapers off from top to bottom in relation to the duct vertical direction H.

The delimiting elements 10a, 10b are in each case flush with the face of an outside wall portion 14a, or 14b, respectively, of the upper clamping leg 3b in the duct transverse direction Q. The delimiting elements 10a, 10b furthermore have in each case one inside wall portion 15a, or 15b, respectively. These wall portions 15a, 15b in relation to the duct transverse direction Q are in each case supported in relation to a sliding wall portion 16a, or 16b, respectively. The two sliding wall portions 16a, 16b are outside end faces of the clamping element 8a that is disposed on the lower clamping leg 3a. In a movement of the clamping legs 3a, 3b between the opening position and the clamped-shut position the delimiting elements 10a, 10b by way of the inside wall portions 15a, 15b thereof are supported so as to be movable in a sliding manner in relation to the sliding wall portions 16a, 16b. Lateral guiding of the clamping legs 3a, 3b is achieved in this way, and canting of the hose clamp 1 is counteracted.

As can be furthermore seen, in particular by means of FIGS. 1 to 3, the extent of the delimiting elements 10a, 10b in the duct vertical direction H corresponds at least to the height of the passage duct 7 between the clamping faces 9a, 9b when the clamping legs 3a, 3b are in the opening position. In other words, the delimiting elements 10a, 10b are designed in such a manner that the passage duct 7 in the region of the clamping faces 9a, 9b by means of the delimiting elements 10a, 10b is at all times closed in the duct transverse direction Q and to this extent is closed independently of the position of the clamping legs 3a, 3b.

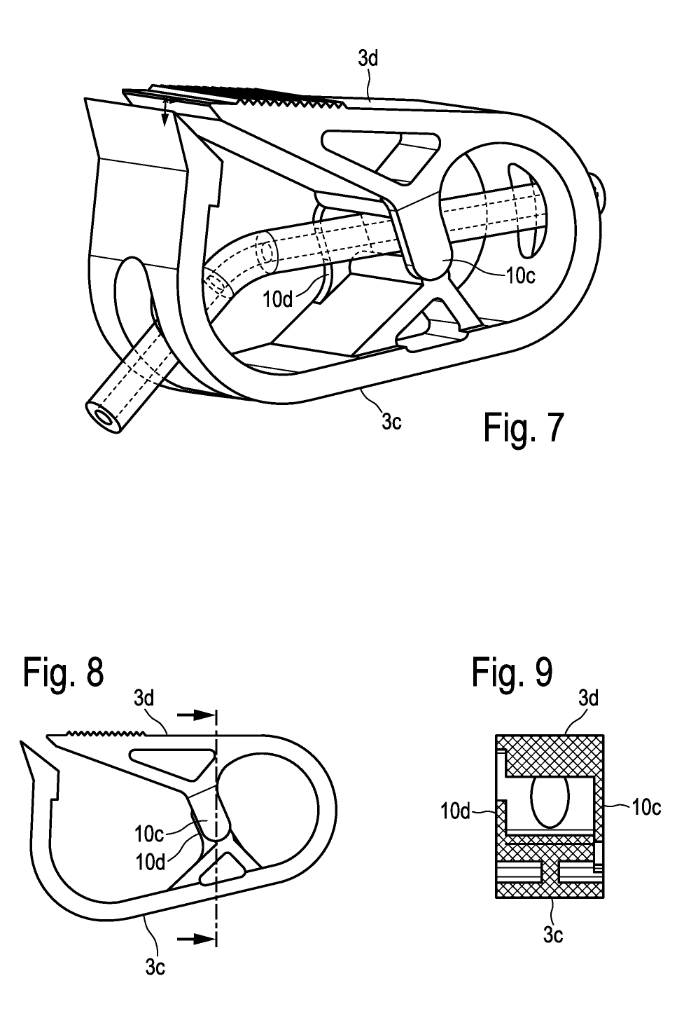

The hose clamp according to FIGS. 7 to 9 corresponds largely to the hose clamp 1 according to FIGS. 1 to 6. In order for repetitions to be avoided, reference is therefore additionally made to the description pertaining to the hose clamp according to FIGS. 1 to 6. The points of differentiation of the hose clamp according to FIGS. 7 to 9 will be discussed hereunder, wherein FIG. 9 shows a sectional illustration along the section line in FIG. 8.

The hose clamp according to FIGS. 7 to 9 has an upper clamping leg 3d and a lower clamping leg 3c. Each clamping leg 3c, 3d is in a laterally external manner assigned in each case one delimiting element 10c, 10d, the latter being mutually diametrically opposite and in the transverse direction being in each case offset so as to protrude toward the other clamping leg. The delimiting elements 10c, 10d are consequently disposed in an alternating manner on the upper clamping leg 3d or on the lower clamping leg 3c. This means that the delimiting element 10c according to the illustration as per FIGS. 7 to 9 is molded so as to be integral to the upper clamping leg 3d, while the other delimiting element 10d is molded on the lower clamping leg 3c. The alignment of the delimiting elements 10c, 10d, and the respective overlap in the region of clamping faces corresponds to that of the embodiment as per FIGS. 1 to 6.

What is claimed:

1. A hose clamp for clamping shut a medical hose, the hose clamp comprising:
two clamping legs which are mutually movable about a pivot axis between an opening position and a clamped-shut position and in the clamped-shut position are mutually latched by a latch;
a passage duct for receiving the hose to be clamped shut, wherein the passage duct along a duct longitudinal direction extends between two passage openings and in relation to a duct vertical direction on an upper side and a lower side is in each case at least in portions covered by the clamping legs; and
two clamping elements which are disposed on the upper side and the lower side of the passage duct and which have in each case one clamping face, said clamping faces by way of a movement of the clamping legs being mutually actuatable to the clamped-shut position in such a manner that the passage duct in the duct vertical direction is capable of being constricted and the hose that is receivable in the passage duct is capable of being clamped shut,
wherein, in relation to a duct transverse direction in each case one delimiting element is disposed on opposite transverse sides of the passage duct,
wherein the delimiting elements are designed in such a manner that the passage duct in a region of the clamping faces is closed in the duct transverse direction and said passage duct in the duct transverse direction is at least in portions released both ahead of and behind the clamping faces in relation to the duct longitudinal direction, such that the hose to be clamped shut, when viewed in a lateral direction, remains uncovered by the delimiting elements across as wide a region of the passage duct as possible.

2. The hose clamp according to claim 1, wherein the delimiting elements in the duct longitudinal direction extend in each case across a length of the clamping elements.

3. The hose clamp according to claim 1, wherein the delimiting elements in the duct longitudinal direction extend in each case across at most double a length of the clamping faces.

4. The hose clamp according to claim 1, wherein the delimiting elements are in each case configured in the form of a strip that is oriented substantially in the duct vertical direction.

5. The hose clamp according to claim 1, wherein the delimiting elements have in each case one front edge and one rear edge which run in a mutually parallel or wedge-shaped manner.

6. The hose clamp according to claim 1, wherein the delimiting elements in the duct transverse direction are in each case flush with the face, or recessed, in relation to in each case one outside wall portion of one of the clamping legs.

7. The hose clamp according to claim 1, wherein the delimiting elements are disposed on the upper one of the clamping legs in the duct vertical direction.

8. The hose clamp according to claim 1, wherein the delimiting elements in an alternating manner are disposed on the upper clamping leg or the lower clamping leg.

9. The hose clamp according to claim 1, wherein the delimiting elements are configured so as to be integral to the clamping legs and to the clamping elements.

10. The hose clamp according to claim 1, wherein an extent of the delimiting elements in the duct vertical direction corresponds to at least a height of the passage duct between the clamping faces when the clamping legs are in the opening position.

11. The hose clamp according to claim 1, wherein the delimiting elements have in each case one wall portion which is in each case supported in the duct transverse direction in relation to one sliding wall portion of one of the clamping legs and/or of the clamping elements and in a sliding manner is movable perpendicular in relation to the duct transverse direction.
